(19) United States
(12) Patent Application Publication
(10) Pub. No.: US 2008/0294190 A1
(43) Pub. Date: Nov. 27, 2008

(54) Adjustable length forceps

(76) Inventor: Terrence R. Young, Erie, CO (US)

Correspondence Address: Tyco Healthcare Group LP, 60 MIDDLETOWN AVENUE, NORTH HAVEN, CT, 06473, US

(21) Appl. No.: 11/805,062

(22) Filed: May 22, 2007

(51) Int. Cl. A61B 17/28

(52) U.S. Cl. 606/205

(57) ABSTRACT

An adjustable hand-held surgical instrument is provided and includes a first elongated member; and a second elongated member pivotally connected to the first elongated member. Each of the elongated members includes a handle portion selectively extendable from the proximal end of the elongated member; and a tool member selectively extendable from the distal end of the elongated member.

### BACKGROUND

1. Technical Field

The present disclosure relates to hand-held surgical instruments, and more particularly, to adjustable length hand-held surgical forceps.

2. Background of Related Art

Hand-held surgical instruments are well known in the art and may be configured for use in myriad of ways. Among the various hand-held instruments that are commercially available are forceps, scissors, pliers, vessel sealers, clip appliers, spreaders, staplers and the like. Many of these instruments include two elongated members connected at a pivot point. The proximal ends of the elongated members form a handle configured to be gripped with at least a finger and a thumb of the same hand. The distal ends of the elongated members form the functional end of the instrument, for example, with forceps, the distal ends from a pair of jaw members for grasping objects therebetween. Hand-held instruments configured in this manner are actuated by squeezing the at least one finger and thumb of the same hand together. Alternatively, the functional distal ends may be actuated by spreading the at least one finger and thumb apart. Either actuation method may be configured to cause either opening or closing of the functional distal ends of the hand-held instruments.

Hand-held instruments are available in a wide range of sizes and configurations for use in various types of procedures and for use by individuals with hands of different sizes. Just as an individual with oversized hands may not feel comfortable working with undersized instruments, an individual with petite hands may feel awkward attempting to manipulate an oversized instrument. Thus, properly sized instruments are important to the comfort of the user and to the facility of manipulation.

Comfort of the user is not the only factor in determining the necessary configuration of an instrument. The depth and/or configuration of the surgery site or the procedure being performed are also of importance in determining the length of an instrument. Procedures performed deep within the body cavity may necessitate the use of instruments of a longer length. However, while accessing the surgery site and while closing the site following completion of the procedure, such instruments of longer length are not necessary, and may be awkward for the surgeon to manipulate. In these instances instruments of a shorter length are preferred. Thus, even within the same procedure, instruments of differing lengths may be desired or even necessary. In the event persons of different hand sizes may need to use the various types of instruments, various sized instruments of longer and shorter lengths may be required.

Providing medical personnel with the proper instruments to perform a procedure is important to a successful completion of that procedure. Unfortunately, to comfortably outfit the medical personnel with the proper instruments may require having multiple instruments of the same type on hand, each of which are sized for different users or for use at different times during the procedure. Requiring more instruments to be used or made available during a procedure, increases the complexity of the logistics of the procedure. Additionally, in the case of reusable instruments, the greater the number of instruments used, the greater the number of instruments that must be cleaned, accounted for throughout the procedure, and sterilized following the procedure.

Therefore, it would be beneficial to have a hand-held surgical instrument that includes one or more selectively adjustable lengthened members and/or jaws configured for adjusting the length and/or configuration of the instrument.

### SUMMARY

The present disclosure relates to adjustable length hand-held surgical forceps. According to an aspect of the present disclosure, such an instrument includes a handle portion configured for operable engagement by a user; a base portion including a proximal end and distal end, said base portion configured to selectively receive said handle portion from said proximal end thereof, wherein a portion of the handle portion is adjustable relative to the base portion; and a tool portion selectively extendable from said distal end of said base portion, wherein a portion of the tool portion is adjustable relative to the base portion.

The base portion may include a first elongated member operably connected to a second elongate member. The first elongated member may be pivotally connected to the second elongate member.

The tool portion may include a jaw member selectively engageable with a respective elongated member.

The instrument may further include at least one adjustment mechanism for selectively extending the handle portion relative to the base portion.

The instrument may still further include one or more adjustment mechanisms for selectively extending the tool portion relative to the base portion.

The at least one adjustment mechanism may include a screw for selectively securing at least one of the handle portion and the tool portion relative to the base portion.

The at least one adjustment mechanism may include a screw for selectively securing at least one of the handle portion and the tool portion relative to the base portion.

The at least one adjustment mechanism may also include a push button member for selectively securing at least one of the handle portion and the tool portion relative to the base portion.

The at least one adjustment mechanism may include a push button member for selectively securing at least one of the handle portion and the tool portion relative to the base portion.

According to another aspect of the present disclosure, an adjustable hand-held surgical instrument is provided and includes a first elongated member; and a second elongated member pivotally connected to the first elongated member. Each of the elongated members includes a handle portion selectively extendable from the proximal end of the elongated member; and a tool member selectively extendable from the distal end of the elongated member.

Each of the first and second elongated members may include at least one adjustment mechanism for selectively securing at least one of the handle portion and the tool portion relative to the base portion.

The handle portions may define ring configured for operable engagement by a user.

The tool member may include a pair of jaw members extending from a respective elongate member for grasping tissue therebetween.

### BRIEF DESCRIPTION OF THE DRAWINGS

The foregoing summary, as well as the following detailed description will be better understood when read in conjunction with the appended figures. For the purpose of illustrating the present disclosure, a preferred embodiment is shown. It is understood, however, that the present disclosure is not limited to the precise arrangement and instrumentalities shown.

FIG. 1A is a perspective view of one illustrative embodiment of an adjustable length forceps according to the present disclosure, in a first or shortened condition;

FIG. 1B is an enlarged view of the indicated area of detail of a portion of the adjustable length forceps of FIG. 1A;

FIG. 2A is a perspective view of the adjustable length forceps of FIG. 1A, in a second or extended condition;

FIG. 2B is an enlarged perspective view of the indicated area of detail of a portion of the adjustable length forceps of FIG. 2A;

FIGS. 3A-3C are enlarged cross-sectional side views of an adjustment mechanism of the adjustable length forceps of FIGS. 1A-2B, in a first or shortened condition (FIGS. 3A and 3B) and in a second or extended condition (FIG. 3C);

FIGS. 4A and 4C-4D are enlarged cross-sectional side views of an alternate embodiment of an adjustment mechanism of the adjustable length forceps of FIGS. 1A-2B, in a first or shortened condition (FIGS. 4A and 4C), and in a second or partially extended condition; and

FIG. 4B is a cross-sectional top view of the adjustment mechanism of FIG. 4A taken along line 4B-4B.

### DETAILED DESCRIPTION OF EMBODIMENTS

The foregoing summary, as well as the following detailed description will be better understood when read in conjunction with the appended figures. For the purpose of illustrating the present disclosure, various embodiments are shown. It is understood, however, that the present disclosure is not limited to the precise arrangement and instrumentalities shown.

As shown in the drawings and described throughout the following description, as is traditional when referring to relative positioning on an object, the term “proximal” refers to the end of the apparatus which is closer to the user and the term “distal” refers to the end of the apparatus which is further from the user.

The embodiments of the present disclosure will be described as they relate to hand-held surgical forceps, however, it is envisioned that the aspects of the present disclosure may be incorporated into any number of hand-held devices, including but not limited to, clip appliers, vessel sealers, scissors, pliers, staplers and the like.

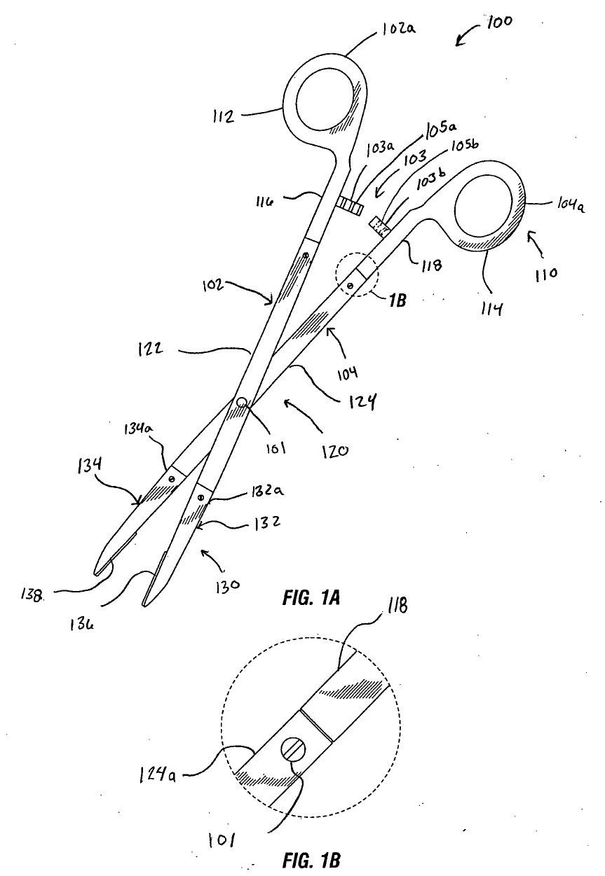

Referring to FIGS. 1A-4B, an illustrative embodiment of the presently disclosed adjustable length forceps is illustrated therein and generally designated as adjustable length forceps 100. Forceps 100 includes first and second elongated members 102, 104. First and second elongated members 102, 104 are substantially mirror images of one another. First and second elongated member 102, 104 are connected at a pivot point by a pivot pin 101. First and second elongate members 102, 104 together form a handle portion 110, a base portion 120 and a tool portion 130.

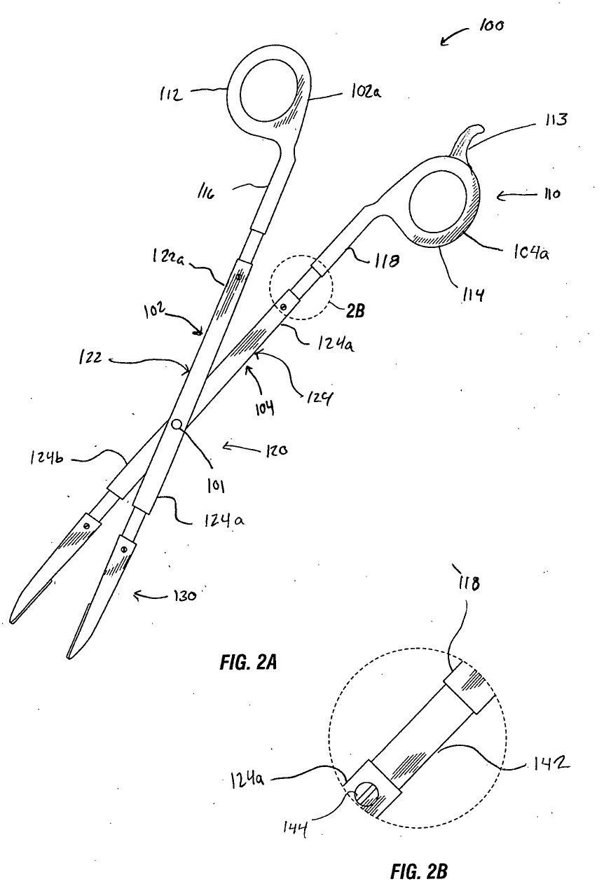

Proximal ends 102a, 104a of first and second elongated members 102, 104, respectively, form handle portion 110 of forceps 100. Handle portion 110 may form loops or rings 112, 114, as shown, or alternatively handle portion 110 may be knurled or may include indents, ridges or the like configured for facilitating grasping and handling by a user. As shown, rings 112, 114 form substantially similar openings for receiving a finger or thumb, respectively, of the same hand. Rings 112, 114 may however, be configured to receive more than one finger. Rings 112, 114 instead form an open configuration or may include a finger rest or tab portion 113 (see FIG. 2A). Rings 112, 114 may further be coated with rubber, plastic or other suitable materials to increase the graspability of handle portion 110. For larger hand-held instruments, it is envisioned that proximal ends 102a, 104a of first and second elongated members 102, 104, respectively, may be configured for engagement with two hands.

Handle portion 110 includes extension members 116, 118 extending from respective rings 112, 114. Extension members 116, 118 are configured to operably connect handle portion 110 with base portion 120, as will be described below. Extension members 116, 118 may be of any length and are preferably of the same length. Rings 112, 114 and extension members 116, 118 may be composed of like or similar materials, or may instead be composed of different material. Rings 112, 114 and respective extension members 116, 118 may be integrally formed with one another, or may be securely or releasably joined with one another. In this manner, rings 112, 114 may be replaced if they become damaged, warn or if a user with different preferences operates forceps 100.

Handle portion 110 may further include a closure mechanism 103 for maintaining first and second elongated members 102, 104 in a predetermined, spaced apart relation on in an approximated or clamped relation. Closure mechanism 103 may also be configured to create a predetermined closure pressure between a first and a second jaw member 132, 134. A variety of closure mechanisms are known in the art and may be incorporated into adjustable length forceps 100. Closure mechanism 103, as shown, includes first and second pawl members 103a, 103b formed on first and second extension members 116, 118, respectively, or may be formed on first and second elongated members 102, 104, respectively. Pawl members 103a, 103b include teeth 105a, 105b, respectively. Pawl members 103a, 103b are positioned opposite one another and are configured such that teeth 105a, 105b selectively engage one another as first and second elongated members 102, 104 are pivoted towards one another. As noted above, closure mechanism 103 may be configured to apply a predetermined pressure between first and second jaw members 132, 134, or in the alternative, to create a predetermined gap therebetween. In an alternate embodiment, closure mechanism 103 may be positioned between first and second elongated members 102, 104 within base portion 120.

Base portion 120 of first and second elongated members 102, 104 includes first and second base members 122, 124. First and second base member 122, 124 form substantially rectangular tubular bodies connected to one another by pivot pin 101. First and second base members 122, 124 may be of any length and may be pivotally connected anywhere along the length thereof. First and second base members 122, 124 include proximal ends 122a, 124a and distal ends 122b, 124b, respectively. Proximal ends 122a, 124a of first and second base members 122, 124 may be configured for selective adjustable engagement with extension members 116, 118 of handle portion 110, as will be discussed below. Distal ends 122b, 124b of first and second base members 122, 124 may be configured for selective adjustable engagement with a first and second jaw member 132, 134 of a tool portion 130.

Tool portion 130 of forceps 100, includes first and second jaw members 132, 134. First and second jaw members 132, 134 include proximal ends 132a, 134a, respectively. First and second jaw members 123, 134 are configured for adjustable engagement with distal ends 122b, 124b of first and second base members 122, 124, respectively. First and second jaw members 132, 134 each further includes grasping surfaces 136, 138. Grasping surfaces 136, 138 may be configured for grasping tissue, sutures, staples and like therebetween. As discussed above, tool portion 130 is described herein relates to hand-held surgical forceps, however, it is understood that the aspects of the present disclosure may be incorporated into any hand-held instrument.

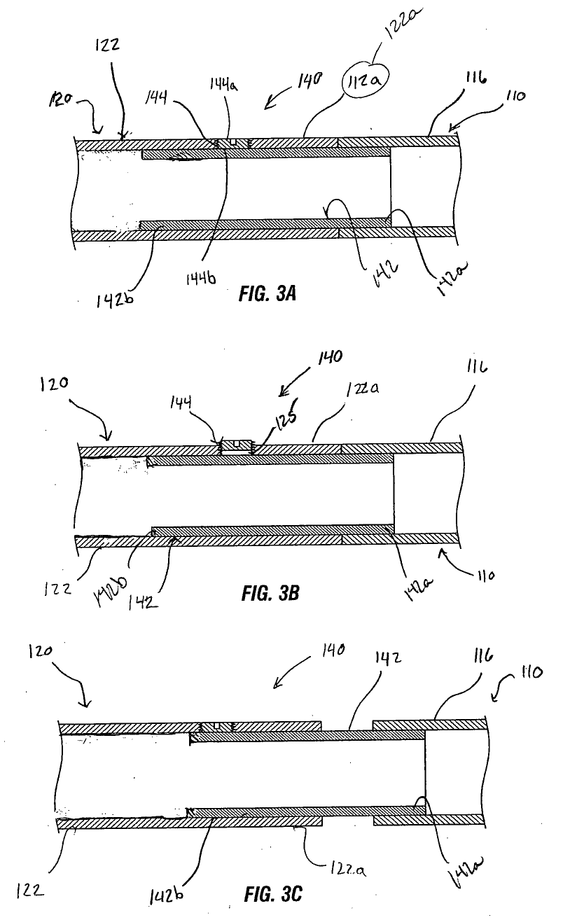

As discussed above, handle portion 110 and tool portion 130 of forceps 100 may be adjustably engaged with base portion 120. First and second elongated members 102, 104 include an adjustment mechanism 140 (FIGS. 3A-3C) for selectively adjusting an overall length of forceps 100.

Referring initially to FIGS. 1A and 1B, forceps 100 are shown in a first or shortened condition. In the shortened condition, extension members 116, 118 of handle portion 110 abut proximal ends 122a, 124a of first and second base members 122, 124, respectively. Also in this first or shortened condition, proximal ends 132a, 134a of first and second jaw members 132, 134 abut distal ends 122b, 124b of first and second base members 122, 124.

Referring now to FIGS. 2A and 2B, forceps 100 are shown in a second or extended condition. In the extended condition extension members 116, 118 of handle portion 110 are in a spaced apart relationship with proximal ends 122a, 124a of first and second base members 122, 124. Also in a second or extended condition, proximal ends 132a, 134a of first and second jaw members 132, 134 are in a spaced apart relationship with distal ends 122b, 124b of first and second base members 122, 124. As will be described in more detail below, adjustment mechanism 140 (FIGS. 3A-3C) may be configured to adjust the overall length of forceps 100 from the first or shortened condition (FIGS. 1A and 1B) to the second or extended condition (FIGS. 2A and 2B) and to any necessary or desired length in between. One or both of the connections between handle portion 110 and base portion 120 and/or one or both of the connections between tool portion 130 and base portion 120 may be selectively extended.

Turning now to FIGS. 3A-3C, an embodiment of adjustment mechanism 140 is shown with handle portion 110 and base portion 120 of forceps 100 in the first or shortened condition (FIGS. 3A and 3B) and in the second or extended condition (FIG. 3C). Adjustment mechanism 140 will be described as it relates to the connection between extension member 116 and proximal end 122a of first base member 122. As discussed above, the connections between handle portion 110 and base portion 120 and the connections between base portion 120 and tool portion 130 may be substantially identical in form and function, or may instead incorporate alternative embodiments of adjustment mechanism 140 therebetween.

Adjustment mechanism 140 includes a shaft 142 having proximal and distal ends 142a, 142b, respectively. Shaft 142 of adjustment mechanism 140 may be formed of any suitable length. Proximal end 142a is configured for secure engagement within extension member 116 of handle portion 110. Proximal end 142a of shaft 142 may be secured within extension member 116 with any known means, including but not limited to adhesives, bonding, welding, and crimping. Shaft 142 may instead be integrally formed with extension member 116. In this manner, distal end 142b of shaft 142 extends from extension member 116. Distal end 142b of shaft 142 is configured to be slidingly received within proximal end 122a of first base member 122.

Adjustment mechanism 140 further includes a mixing member or screw 144. Screw 144 is configured to be received within a threaded opening 125 (see FIG. 3B) formed in proximal end 122a of first base member 122. Screw 144 includes a tool engaging top surface 144a and a shaft engaging bottom surface 144b. Alternatively, top surface 144a of screw 144 may be configured to include a tab or lever such that screw 144 may be tightened or loosened by hand. As will be described below, the tightening of screw 144 within opening 125 causes bottom surface 144b to frictionally engage a surface of shaft 142.

Referring to FIG. 3A, in the first or shortened condition of forceps 100, extension member 116 abuts proximal end 122a of first base member 122. Shaft 142, securely received with extension member 116, extends into proximal end 122a of first base member 122. Screw 144 is shown tightly secured against the surface of shaft 142. In this manner, handle portion 110 and base portion 120 are frictionally engaged with one another. Alternatively, distal end 142b of shaft 142 may include indents, tabs or openings (not shown) for more secured engagement with bottom surface 144b of screw 144. With reference to FIG. 3B, with forceps 100 still in a first or shortened condition, screw 144 is shown retracted or spaced away from the surface of shaft 142. Thus, distal end 142b of shaft 142, extending distally from within extension member 116, is no longer secured with proximal end 122a of first base member 122. In this manner, distal end 142b of shaft 142 may be slidingly withdrawn from within proximal end 122a of first base member 122.

Turning now to FIG. 3C, adjustment mechanism 140 and, in turn, forceps 100, is shown in a second or extended condition. Distal end 142b of shaft 142 has been at least partially withdrawn from within proximal end 122a of first base member 122. In this manner, handle portion 110 is extended from base portion 120, thereby increasing the overall length of first elongated member 102 and thus the overall length of forceps 100. When in the second or extended condition screw 144 is or may be retightened against the surface of shaft 142, thereby lockingly securing extension member 116 relative to first base member 122. Shaft 142 of adjustment mechanism 140 may include a lip, stop, tab or the like (not shown) for preventing complete withdrawal of shaft 142 from first base member 122. Alternatively, shaft 142 may be configured to be completely withdrawn from with first base member 122 such that handle portion 110 may be changed or replaced.

While adjustment mechanism 140 has been described with extension member 116 being extended relative to first base member 122, it is envisioned that adjustment mechanism 140 may instead be configured such that first base member 122 is extended relative to extension member 116. In this manner, distal end 142b of shaft 142 would be securely received within proximal end 122a of first base member 122 and proximal end 144b of shaft 142 would be slidingly received within extension member 116. Extension member 116 would then define opening 125 therein for receiving screw 144.

In an alternative embodiment, forceps 100 may include more than one adjustment mechanisms 140 laterally disposed about proximal end 122a of first base member 122. In this manner, distal end 142b of shaft 142 may be more securely received within proximal end 122a of first base member 122. In yet another embodiment, distal end 142b of shaft 142 may simply be frictionally received within proximal end 122a of first base member 122.

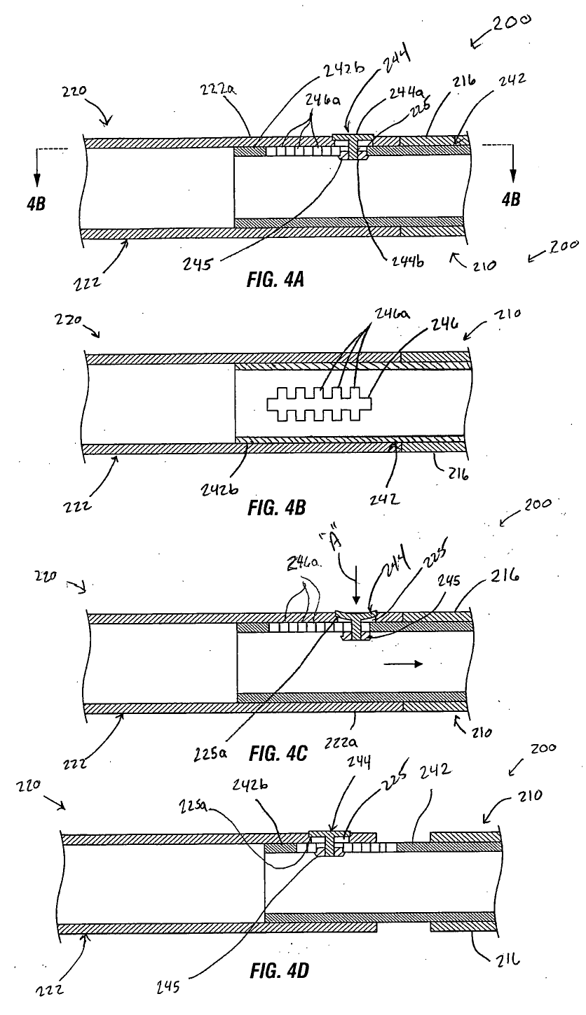

Referring now to FIGS. 4A-4D, in an alternate embodiment of the present disclosure forceps 200 may include an adjustment mechanism 240. As provided above, adjustment mechanism will be described in relation to the connection between extension member 216 and first base member 222, however, any of the connections between handle portion 110 and base portion 120 or base portion 120 and tool portion 130 may be configured as described herein. Additionally, adjustment mechanism 240 will be described in detail only as it relates to the differences with adjustment mechanism 140 described hereinabove.

As seen in FIGS. 4A-4D, adjustment mechanism 240 includes a push button or actuation/locking member 244 configured for releasably securing distal end 242b of shaft 242 within proximal end 222a of first base member 222.

Referring initially to FIGS. 4A and 4C-4D, proximal end 222a of first base member 222 defines an opening 225 for receiving button member 244. Proximal end 222a of first base member 222 further defines a shoulder 225a extending about opening 225 for receiving button member 244 thereagainst. Button member 244 includes a top surface 244a configured to engage shoulder 225a and span opening 225. Top portion 244a of button member 244 is configured such that downward pressure thereon (as indicated by arrow “A” of FIG. 4C) deflects button member 244 within opening 225. Button member 244 further includes a bottom portion 244b configured to be received within a slot 246 formed in distal end 242b of shaft 242. Bottom portion 244b of button member 244 further includes tab portions 245 for engaging distal end 242b distal of shaft 242.

Referring now to FIG. 4B, as discussed above distal end 242b of shaft 242 defines slot 246 for receiving bottom portion 244b of button member 244. Distal end 242b of shaft 242 further defines grooves 246a extending from slot 246 for releasably engaging tab portions 245 of button member 244. Grooves 246a may be spaced a predetermined distance from one another such that handle portion 110 may be extended predetermined increments relative to base portion 220. Distal end 242b of shaft 242 may further include any number of grooves 246a for releasable engaging tab portions 245.

In use, referring initially to FIG. 4A, adjustment mechanism 240 and, in turn, the forceps, is shown in a first or shortened condition. Button member 244 is shown in an initial, non-depressed state. As shown, distal end 242b of shaft 242 extends from handle portion 210 is completely received within proximal end 222a of first base portion 222. Tab portion 245 of button member 244 is securely received within a groove 246a formed in distal end 242b of shaft 242. In this manner, handle portion 210 is lockingly engaged with base portion 220.

With reference now to FIG. 4C, still in a first or shortened condition, button member 244 is shown in a pushed or depressed state. By depressing top portion 244a of button member 244, as indicated by arrow “A”, bottom portion 244b, and more particularly, tab portion 245 are extended from within groove 246a formed in distal end 242b of shaft 242, thereby withdrawing distal end 242b of shaft 242 relative to proximal end 222a of first base member 222 and unlocking shaft 242 relative to base portion 222. As so configured, handle portion 210 may be extended relative to base portion 220.

Turning now to FIG. 4D, adjustment mechanism 240 is shown in a second or partially extended condition. Distal end 244b of shaft 244 has been partially withdrawn from within proximal end 222a of first base member 222. In this manner, handle portion 210 is extended from and relative to base portion 220, thereby increasing the overall length of first elongated member 202, and thus, the overall length of forceps 200. In the second or extended condition, button member 244 released thereby allowing button member 244 to return to its initial, non-depressed state. Tab portion 245 is thus, re-received within one of grooves 246a formed in distal end 244b of shaft 244, thereby lockingly securing extension member 216 relative to first base member 222.

Thus, it should be understood that various changes in form, detail and operation of the adjustable length forceps of the present disclosure may be made without departing from the spirit and scope of the present disclosure.

What is claimed is:

1. An instrument comprising:
a handle portion configured for operable engagement by a user;
a base portion including a proximal end and distal end, said base portion configured to selectively receive said handle portion from said proximal end thereof, wherein a portion of the handle portion is adjustable relative to the base portion; and
a tool portion selectively extendable from said distal end of said base portion, wherein a portion of the tool portion is adjustable relative to the base portion.

2. The instrument of claim 1, wherein said base portion includes a first elongated member operably connected to a second elongate member.

3. The instrument of claim 2, wherein said first elongated member is pivotally connected to said second elongate member.

4. The instrument of claim 2, wherein said tool portion includes a jaw member selectively engageable with a respective elongated member.

5. The instrument of claim 1, further including at least one adjustment mechanism for selectively extending said handle portion relative to said base portion.

6. The instrument of claim 1, further including one or more adjustment mechanisms for selectively extending said tool portion relative to said base portion.

7. The instrument of claim 5, wherein the at least one adjustment mechanism includes a screw for selectively securing at least one of said handle portion and said tool portion relative to said base portion.

8. The instrument of claim 6, wherein the at least one adjustment mechanism includes a screw for selectively securing at least one of said handle portion and said tool portion relative to said base portion.

9. The instrument of claim 5, wherein the at least one adjustment mechanism includes a push button member for selectively securing at least one of said handle portion and said tool portion relative to said base portion.

10. The instrument of claim 6, wherein the at least one adjustment mechanism includes a push button member for selectively securing at least one of said handle portion and said tool portion relative to said base portion.

11. An adjustable hand-held surgical instrument comprising:
a first elongated member; and
a second elongated member pivotally connected to said first elongated member, wherein each of said elongated members include:
a handle portion selectively extendable from said proximal end of said elongated member; and
a tool member selectively extendable from said distal end of said elongated member.

12. The adjustable hand-held surgical instrument of claim 9, wherein each of said first and second elongated members includes at least one adjustment mechanism for selectively securing at least one of said handle portion and said tool portion relative to said base portion.

13. The adjustable hand-held surgical instrument of claim 9, wherein said handle portions define rings configured for operable engagement by a user.

14. The adjustable hand-held surgical instrument of claim 9, wherein said tool member includes a pair of jaw members extending from a respective elongate member for grasping tissue therebetween.
